(19) United States
(12) Patent Application Publication
(10) Pub. No.: US 2026/0207492 A1
(43) Pub. Date: Jul. 23, 2026

(54) READY-TO-USE SODIUM BICARBONATE INJECTION AND METHODS

(71) Applicant: Nivagen Pharmaceuticals, Inc., Sacramento, CA (US)

(72) Inventors: Niravbhai Jayantibhai Patel, Sacramento, CA (US); Robert Miller, Sacramento, CA (US); Dasaradhi Lakkaraju, Sacramento, CA (US); Jay Shukla, Sacramento, CA (US)

(73) Assignee: Nivagen Pharmaceuticals, Inc., Sacramento, CA (US)

(21) Appl. No.: 19/035,243

(22) Filed: Jan. 23, 2025

(51) Int. Cl. A61K 9/08; A61K 9/00; A61K 33/00; A61K 47/02; A61K 47/26

(52) U.S. Cl. CPC A61K 9/08; A61K 9/0019; A61K 33/00; A61K 47/02; A61K 47/26

(57) ABSTRACT

Disclosed and described is a ready-to-administer parenteral liquid formulations comprising sodium bicarbonate or a pharmaceutically acceptable salt thereof, methods for preparing such formulations, methods for using such formulations, and polymeric infusion container filled with such formulations.

### TECHNICAL FIELD

The present invention relates to a ready-to-use, or ready-to-administer, injectable liquid formulation comprising sodium bicarbonate, a method for preparing such formulation, and a method for using such formulation. The ready-to-administer sodium bicarbonate formulation can be provided in a polymeric infusion container.

### BACKGROUND

Sodium bicarbonate (NaHCO₃) injection is a known medical treatment used primarily to manage conditions of metabolic acidosis and certain types of drug overdoses, among other clinical indications. It is a sterile solution of sodium bicarbonate in water and is commonly administered intravenously (IV).

Sodium bicarbonate has been used for medical purposes since the 19th century, initially for its antacid properties. Over time, its role expanded to more critical and life-saving applications, especially in the management of acid-base disorders. The development of injectable formulations allowed for more precise and rapid intervention in emergency and critical care settings.

Sodium bicarbonate is an alkaline substance that, when introduced into the bloodstream, acts as a buffering agent. It neutralizes excess acid in the body, thereby raising blood pH and correcting metabolic acidosis. Metabolic acidosis occurs when there is an excess of hydrogen ions (H⁺) in the blood, leading to a decrease in pH. Sodium bicarbonate, by dissociating into sodium (Na⁺) and bicarbonate (HCO₃⁻) ions, buffers the acid, thereby reducing acidity.

Sodium bicarbonate injection is used in several clinical settings, including
Metabolic Acidosis, when the blood becomes too acidic due to conditions like renal failure, diabetic ketoacidosis, shock, or lactic acidosis.
Drug Overdose, such as overdose of tricyclic antidepressants (TCAs) and aspirin (salicylates), where the bicarbonate helps to alkalinize the urine and blood to enhance elimination of the drug and reduces its toxic effects on the heart and other organs.
Cardiac Arrest and Severe Arrhythmias, where acidosis has developed during resuscitation efforts, such as in cardiac arrest, and can improve myocardial function, and reduce the risk of arrhythmias.
Chronic Kidney Disease (CKD), where sodium bicarbonate can counteract metabolic acidosis that may accompany CKD.
Hyperkalemia, where sodium bicarbonate can help reduce high potassium levels (hyperkalemia) by driving potassium into cells.

Currently, no ready-to-use (RTU) injectable form of sodium bicarbonate solution is available on the market due to instability of the solution in low concentrations. Currently available solutions are offered at concentrations of 4.2%, 7.5% and 8.4%, each of which can require dilution to a lower concentration prior to administration. For example, the Package Insert for Sodium Bicarbonate Injection, USP, marketed by Exela Pharma Sciences, LLC, provides (Table 1):

TABLE 1

| | | | | | mEq/ | |
|---|---|---|---|---|---|---|
| | | | | | size | |
| | | mg/mL | mEq/mL | mEq/mL | Container | |
| NDC Code: | Conc % | (NaHCO₃) | (Na⁺) | (HCO₃⁻) | (mL) | mOsmol |
| 51754-5001-5 | 8.4 | 84 | 1 | 1 | 50/50 | 2/mL |
| 51754-5011-4 | 8.4 | 84 | 1 | 1 | 10/10 | 2/mL |
| 51754-5002-5 | 7.5 | 75 | 0.9 | 0.9 | 44.6/50 | 1.79/mL |
| 51754-5012-4 | 4.2 | 42 | 0.5 | 0.5 | 5/10 | 1/mL |

While sodium bicarbonate can be a valuable and critical tool in clinical medicine, currently marketed sodium bicarbonate products must be used with caution due to the risk of complications and potential overuse and overdose, particularly in patients with underlying kidney or heart conditions. Complications resulting from excessive use of sodium bicarbonate can include metabolic alkalosis, electrolyte imbalance, or fluid overload, and require careful monitoring of the patient when administered.

Currently marketed formulations are also hyperosmotic (i.e. 2 mOsmol/mL, or about 2000 mOsmol/L). Hence, appropriate caution is required during drug administration. As clearly noted in the package insert for such products: “Caution should be observed in emergencies where very rapid infusion of large quantities of bicarbonate is indicated. Bicarbonate solutions are hypertonic and may produce an undesirable rise in plasma sodium concentration in the process of correcting the metabolic acidosis.”

In addition, currently available sodium bicarbonate products provided in glass vials have been recently recalled due to particulate matter such as silicone or glass particles found in products sold in glass vials. Glass vials are the primary source of Aluminum. High amounts of aluminum, exceeding acceptable limits, can also be present in these products, making such products a risk for administration to patients with renal impairment and younger patients, especially neonates. With respect to levels of aluminum it should be recognized that while most currently used products are below the maximum allowed aluminum level as determined by the Federal Drug Administration (FDA), some of these aluminum concentrations may nevertheless be toxic in renally impaired patients. Moreover, many of the known concentrated compositions include varying amounts of sodium and may not provide a proper ratio of these critical components.

What is needed is a stable, ready-to-use (RTU) injectable solution comprising sodium bicarbonate provided in a polymeric infusion bag at a concentration about three to six times lower than injectable sodium bicarbonate solution currently available in glass vials.

In addition, sodium bicarbonate solutions having low aluminum content are needed for safe administration to patients of all ages. Sodium bicarbonate solutions having low aluminum content are advantageous for safe administration to patients of all ages. Sodium bicarbonate solutions having low aluminum content are especially advantageous for safe administration to younger patients, age 12 or younger, and can be safely administered to children age 6 or younger, including to neonates.

These desired properties and other advantages are realized by the sodium bicarbonate solution of the subject invention, described and exemplified herein.

### SUMMARY OF THE INVENTION

The subject invention is directed to stable, ready-to-use (RTU), or ready-to-administer, parenteral liquid composition for injection. The composition is a formulation comprising about 1% to about 1.5% sodium bicarbonate. The ready-to-administer formulation can advantageously be sterilized and contained within a polymeric infusion bag for storage and for administration directly from the polymeric infusion bag. The composition of the subject invention is stable upon long-term storage at room temperature, i.e., from about 20° C. to about 25° C.

In one embodiment, the invention is a ready-to-administer parenteral liquid formulation comprising sodium bicarbonate in water for injection. In another embodiment, the ready-to-administer parenteral liquid formulation comprises sodium bicarbonate and dextrose in water for injection. Sodium bicarbonate is provided at a purity of at least 95%, preferably greater than 99%, and more preferably about 99.9%. Dextrose is preferably provided at 5% of the total volume.

Preferably, the liquid formulation of the invention has a pH between 7.0 and 8.5, and more preferably between 7.0 and 7.9. The liquid can be sterilized and contained and stored in a polymeric infusion container. Advantageously, this described liquid formulation is stable for at least 6 months when stored at 25° C.±2° C. and 40% relative humidity (RH)±5% RH. A stable liquid formulation according to the invention can retain at least 95% w/w of the sodium bicarbonate after storage at 25° C.±2° C. and not more than 40% RH for at least 6 months. More preferably, a stable liquid formulation according to the invention can retain at least 98% w/w of the sodium bicarbonate after storage at 25° C.±2° C. and not more than 40% RH for at least 6 months.

The liquid formulation of the invention comprising sodium bicarbonate (without dextrose) is isotonic, having an osmolality of from about 250 mOsmol/kg to about 300 mOsmol/kg, and more specifically about 280 mOsmol/kg. The liquid formulation of the invention comprising sodium bicarbonate with dextrose is hyperosmotic, having an osmolality of from about 500 mOsmol/kg to about 600 mOsmol/kg, and more specifically about 550 mOsmol/kg.

In one preferred embodiment, the liquid formulation of the invention comprises from about 10.0 mg/mL to about 15.0 mg/mL of sodium bicarbonate. In one embodiment, a stable liquid formulation according to the invention comprises about 11.0 mg/mL to about 14 mg/mL of sodium bicarbonate. In one embodiment, a stable liquid formulation according to the invention comprises about 12.0 mg/mL to about 13 mg/mL of sodium bicarbonate. In one embodiment, a stable liquid formulation according to the invention comprises 12.6 mg/mL (1.26%).

In one embodiment, the pharmaceutical product of the invention can comprise a sodium bicarbonate solution, wherein the solution includes less than 25 mcg/L aluminum. Sodium bicarbonate solutions having low aluminum content, e.g., less than 25 mcg/L aluminum can advantageously be safe for administration to patients of all ages. Sodium bicarbonate solutions having less than 25 mcg/L aluminum content are especially advantageous for safe administration to younger patients, age 12 or younger, and can be safely administered to children age 6 or younger, including to neonates.

A method of the invention comprises providing a ready-to use liquid formulation comprising sodium bicarbonate, and parenterally administering the liquid sodium bicarbonate formulation to a patient in need thereof for treating acidosis. In a preferred embodiment, The formulation is administered to a patient suffering from moderate to severe acidosis.

In one preferred embodiment, the invention comprises an administration kit comprising a liquid sodium bicarbonate formulation described herein, contained within a polymeric infusion container. The liquid formulation of the invention can be aseptically sterilized and placed in the polymeric infusion container, and can provide stability for the liquid formulation for about 6 months or more. The formulation comprising sodium bicarbonate without dextrose can be terminally sterilized in the infusion bag. The terminal sterilization can be achieved by autoclave.

In one aspect, the invention comprises an aseptically sterilized solution comprising 1.26% sodium bicarbonate packaged in a ready-to-administer (RTA) intravenous (IV) bag. In another embodiment, the invention comprises a terminally sterilized solution comprising 1.26% sodium bicarbonate, without dextrose, in a ready-to-administer (RTA) intravenous (IV) bag.

The invention further includes a method for making the ready-to-administer parenteral liquid formulations described herein. The method comprises preparing an aseptically sterilized bulk solution comprising sodium bicarbonate, and aseptically filling the bulk solution into a polymeric infusion container. Alternatively, the bulk solution comprising sodium bicarbonate without dextrose can be filled within a polymeric infusion container and terminally sterilized. Terminal sterilization can comprise autoclaving the container filled with the solution.

The invention also includes a method for treating acidosis or certain drug overdoses using the ready-to-administer parenteral liquid formulations described herein. The methods comprise parenterally administering by infusion to a subject in need of treatment of metabolic acidosis or drug overdose treatable by infusion of sodium bicarbonate infusion.

The invention also concerns an administration kit for infusion of a sodium bicarbonate composition to a patient in need thereof. An administration kit according to the invention comprises a polymeric infusion container filled with the ready-to-administer parenteral liquid formulations described herein. The contents of the polymeric infusion containers can be sterilized, without harm to the sodium bicarbonate composition contained therein and without negatively affecting the stability of the sodium bicarbonate composition. When the solution comprises sodium bicarbonate, without dextrose, the solution and container can be terminally sterilized.

These and other objects of the invention are described in detail in the following paragraphs. These objects should not be deemed to narrow the scope of the invention.

### DETAILED DESCRIPTION OF THE INVENTION

This detailed description is intended to describe the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. DEFINITIONS

As used in this specification and the appended claims, unless specified to the contrary, the following terms have the meaning indicated:

The term “about” refers generally to a range of numerical values (e.g., ±5 to 10% of the recited value) that one of ordinary skill in the art would consider equivalent to the recited value. Ranges can be expressed herein as from “about” one particular value, and/or to “about” another particular value. When such a range is expressed, the range is inclusive of the recited values.

The term “aluminum” as used herein refers to both aluminum ions (e.g., Al³⁺) as well as metallic aluminum.

The term “initial” when used in connection with a given parameter such as osmolality, pH, sodium bicarbonate content, or impurities refers to that parameter at the time the sodium bicarbonate composition is filled into the polymeric infusion container under aseptic conditions.

The term “pharmaceutically acceptable” is used adjectivally to mean that the modified noun is appropriate for use as a pharmaceutical product for human use or as a part of a pharmaceutical product for human use.

The terms “ready-to-administer” and “ready-to-use” are interchangeable and mean a composition that does not require dilution or other manipulation, e.g., addition of another substance or diluent, prior to administration from the container in which the composition is provided.

The term “subject” includes humans and other primates as well as other mammals. The term subject includes, for example, a human or mammalian patient suffering from or believed to be suffering from metabolic acidosis, ketoacidosis, or overdose of certain drugs where alkalinization of the blood or urine is determined to be a proper treatment.

The terms “treat”, “treating” and “treatment” refer to a method of alleviating, abrogating, managing, or mediating a condition, disorder, or disease and/or the attendant symptoms thereof.

### B. DRUG SUBSTANCE

Liquid formulations disclosed herein comprise at least one active pharmaceutical ingredient, which is sodium bicarbonate.

In certain embodiments, sodium bicarbonate is present in the liquid formulation in an amount from about 10 mg/mL to about 15 mg/mL of sodium bicarbonate. In certain embodiments, the liquid formulation comprises about 11 mg/mL to about 14 mg/ml of sodium bicarbonate. In certain embodiments, sodium bicarbonate is present in the liquid formulation in an amount from about 12 mg/mL to about 13 mg/mL of sodium bicarbonate. In certain embodiments, sodium bicarbonate is present in the liquid formulation in an amount of 12.6 mg/mL of sodium bicarbonate, USP.

### C. LIQUID FORMULATION

The subject invention comprises a liquid formulation that can be aseptically sterilized and contained and storable in a polymeric infusion container and is stable upon long-term storage. The liquid formulation may be pH adjusted using a gas, such as carbon dioxide (CO₂) bubbled through the composition. In an embodiment comprising sodium bicarbonate without dextrose, the solution contained within the polymeric infusion bag can be terminally sterilized.

A summary of one embodiment of a formulation according to the invention is shown in Table 2 and Table 3.

TABLE 2
Composition for Sodium Bicarbonate Injection 0.15 mEq/mL

| Ingredient | mg/mL | For 5000 mL |
|---|---|---|
| Sodium Bicarbonate USP | 12.6 | 63 g |
| Water for Injection | QS to 1 mL | QS to 5 L |
| Carbon Dioxide gas | Q.S. | Q.s. to adjust the pH |

TABLE 3
Composition for Sodium Bicarbonate in
5% Dextrose Injection 0.15 mEq/ml

| Ingredient | mg/mL | For 5000 mL |
|---|---|---|
| Sodium Bicarbonate USP | 12.6 | 63 g |
| Dextrose Monohydrate | 50 | 250 g |
| Water for Injection | NA | QS to 5 L |
| Carbon Dioxide gas | Q.S. | Q.s. to adjust the pH |

In certain embodiments, the liquid formulation is isotonic.

In certain embodiments, the liquid formulation (without dextrose) has an osmolality in the range from about 250 mOsmol/kg to about 350 mOsmol/kg, alternatively, about from about 250 mOsmol/kg to about 320 mOsmol/kg, or, alternatively, from about 260 mOsmol/kg to about 310 mOsmol/kg. In an exemplary embodiment, the osmolality of the liquid formulation is about 280 mOsmol/kg.

In certain embodiments, the liquid formulation (with dextrose) has an osmolality in the range from about 500 mOsmol/kg to about 600 mOsmol/kg, alternatively, about from about 520 mOsmol/kg to about 580 mOsmol/kg, or, alternatively, from about 525 mOsmol/kg to about 575 mOsmol/kg. In an exemplary embodiment, the osmolality of the liquid formulation is about 550 mOsmol/kg.

In certain embodiments, the liquid formulation has an initial pH in the range of about 7.0 to about 8.5, preferably from about 7.0 to about 7.9, and more preferably about 7.5.

### Preparation of Compositions

A. Dispense the required amount of Sodium Bicarbonate after potency correction.
B. Sodium bicarbonate formulation without dextrose

Detailed below are the steps that were undertaken in the preparation of Sodium Bicarbonate Injection 0.15 m Eq/mL which contains API of (anhydrous) Sodium Bicarbonate USP.
B.1 Preparation of Bulk solution
   B.1.1 Collect 80% of water for Injection into suitable container
   B.1.2 Bubble CO₂ gas at psi (40 psi) for 30 min. During bubbling the container is closed partially.
   B.1.3 Weigh and transfer slowly required quantity of Sodium bicarbonate USP into the above container. Stir the solutions until the API completely dissolves. During the process sparge Carbon Dioxide Continuously
   B.1.4 Make up the volume up to 100% with CO₂ bubbled water. Mix well and check the pH. (target pH Range: 7.0 to 8.5)
   If the pH is not within the range, bubble CO₂ to adjust the pH of solution.
   B.1.5. Filter the bulk solution through 0.2 μm PVDF or PES filter. Bubble CO₂ gas through the filtered solution to maintain pH and also for flushing the headspace of the container. Terminally sterilized the drug product.
C. Sodium bicarbonate formulation with 5% dextrose

Follow above process, except add the required quantity of Dextrose in the solution and avoid terminal sterilization process to make Sodium Bicarbonate in 5% Dextrose Injection.

### Stability Data

Shown in Table 4 and Table 5, below are the results of stability testing for a composition of the subject invention, namely, a 1.26% sodium bicarbonate solution for injection, without dextrose, and a 1.26% sodium bicarbonate solution for injection, with dextrose.

TABLE 4
1.26% Sodium Bicarbonate Injection (without Dextrose)

| | | | 1M- | 1M- | | | | |
|---|---|---|---|---|---|---|---|---|
| | | | 25° C./ | 40° C./ | 3M | 3M | 6M | 6M |
| Test | Specification | Initial | 40% RH | 75% RH | 25/40 | 40/15 | 25/40 | 40/15 |
| Description | Clear | Complies | Complies | Complies | Complies | Complies | Complies | Complies |
| | colorless | | | | | | | |
| | solution, | | | | | | | |
| | free from | | | | | | | |
| | visible | | | | | | | |
| | particles. | | | | | | | |
| pH | Between 7.0 | 7.58 | — | — | 7.62 | 7.56 | 7.93 | 7.95 |
| | and 8.5 | | | | | | | |
| Osmolarity | To be | 269 | 277 | 278 | 279 | 280 | 280 | 278 |
| | reported | | | | | | | |
| | (mOsm/L) | | | | | | | |
| Assay of Sodium | Not less | 99.48% | 100.81 % | 100.81 % | 99.48% | 100.81 % | 100.81 % | 99.48% |
| Bicarbonate by | than 95.0% | | | | | | | |
| Titrimetry | and Not | | | | | | | |
| Each mL contains: | more than | | | | | | | |
| Sodium | 105.0% | | | | | | | |
| Bicarbonate, 12.6 | | | | | | | | |
| mg | | | | | | | | |

Particulate matter A) Visible particles

| | | | | | | | | |
|---|---|---|---|---|---|---|---|---|
| Visual inspection | Should be | Complies | Complies | Complies | Complies | Complies | Complies | Complies |
| | free of | | | | | | | |
| | visible | | | | | | | |
| | particles | | | | | | | |

Particulate matter B) Sub-visible Particles (By Light Obscuration method)

| | | | | | | | | |
|---|---|---|---|---|---|---|---|---|
| ≥10 μm size | Not more | 39.33 | 2.00 | 7.67 | 6.87 | 9.60 | 4.67 | 1.53 |
| particles | than 6000 | | | | | | | |
| | particles per | | | | | | | |
| | container | | | | | | | |
| ≥25 μm size | Not more | 1.33 | 0.33 | 0.00 | 0.47 | 0.93 | 0.13 | 0.00 |
| particles | than 600 | | | | | | | |
| | particles per | | | | | | | |
| | container | | | | | | | |
| Aluminum | NMT 25 μg/L | — | — | — | — | — | <1 μg/L | — |
| content (μg/L) | | | | | | | | |

TABLE 5
1.26% Sodium Bicarbonate in 5% Dextrose Injection

| | | | 1M- | 1M- | | | | |
|---|---|---|---|---|---|---|---|---|
| | | | 25° C./ | 40° .C/ | 3M | 3M | 6M | 6M |
| Test | Specification | Initial | 40% RH | 75% RH | 25/40 | 40/15 | 25/40 | 40/15 |
| Description | Clear | Complies | Complies | Complies | Complies | Complies | Complies | Complies |
| | colorless to | | | | | | | |
| | pale yellow | | | | | | | |
| | solution, | | | | | | | |
| | free from | | | | | | | |
| | visible | | | | | | | |
| | particles. | | | | | | | |
| pH | Between 7.0 | 7.78 | — | — | 7.55 | 7.58 | 7.52 | 7.51 |
| | and 8.5 | | | | | | | |
| Osmolarity | To be | 536 | 554 | 550 | 549 | 551 | 555 | 553 |
| | reported | | | | | | | |
| | (mOsm/L) | | | | | | | |
| Assay of Sodium | Not less | 99.48% | 99.48 % | 100.01 % | 101.4 | 101.4 | 100.1 | 98.8 |
| Bicarbonate by | than 95.0% | | | | | | | |
| Titrimetry | and Not | | | | | | | |
| Each mL contains: | more than | | | | | | | |
| Sodium | 105.0% | | | | | | | |
| Bicarbonate, | | | | | | | | |
| 12.6 mg | | | | | | | | |

Particulate matter A) Visible particles

| | | | | | | | | |
|---|---|---|---|---|---|---|---|---|
| Visual inspection | Should be | Complies | Complies | Complies | Complies | Complies | Complies | Complies |
| | free of | | | | | | | |
| | visible | | | | | | | |
| | particles | | | | | | | |

Particulate matter B) Sub-visible Particles (By Light Obscuration method)

| | | | | | | | | |
|---|---|---|---|---|---|---|---|---|
| ≥10 μm size | Not more | 13.00 | 319.67 | 4.33 | 6.60 | 9.60 | 7.60 | 8.73 |
| particles | than 6000 | | | | | | | |
| | particles per | | | | | | | |
| | container | | | | | | | |
| ≥25 μm size | Not more | 1.00 | 8.33 | 1.33 | 0.53 | 0.93 | 1.40 | 0.93 |
| particles | than 600 | | | | | | | |
| | particles per | | | | | | | |
| | container | | | | | | | |
| Aluminum | NMT | — | — | — | — | — | 1 μg/L | — |
| content (μg/L) | 25 μg/L | | | | | | | |

### D. POLYMERIC INFUSION CONTAINERS

The sodium bicarbonate formulations disclosed herein are ideally ready-to-administer (RTA) formulations for parenteral administration without the need for reconstitution or further dilution. The ready-to-administer formulations are liquid stored in a pharmaceutically acceptable container, for example, an infusion container such as polymeric infusion container (e.g., intravenous (IV) bag).

Exemplary materials for the containers and container components include, but are not limited to, polyolefins, polysulfone, polycarbonate, polypropylene, polyethylene (such as low-density polyethylene (LDPE) or high-density polyethylene (HDPE)), polystyrene, and co-polymers thereof. Preferably, the materials for the container and container components are suitable to withstand aseptic filling and not deform during terminal sterilization. In certain embodiments, the polymeric infusion container comprises a polyolefin/styrene block copolymer, such as the polymeric IV bags manufactured by PolyCine OR Sifra.

The infusion container can be a flexible plastic container, optionally with ports and closure system for storing the liquid formulations. Infusion containers can include other conventional components, for example, connection ports, connector caps or connector disks.

The infusion containers may be sealed with any appropriate closure, such as a twist-off stopper. An exemplary twist-off stopper comprises a polypropylene film, such as Inerta® supplied by Technoflex® or equivalent.

In certain embodiments, the sodium bicarbonate liquid formulation is prepared as a 50 mL fill or a 1000 mL fill packaged in a 500 mL or 1000 mL, respectively, polymeric infusion container, such as a IV bag with an appropriate stopper and over pouch with Al pouch.

An exemplary polymeric infusion container and closure system for sodium bicarbonate is provided in Table 6 below.

TABLE 6

| Component | Description |
|---|---|
| IV Bag | Bag: multilayer Polyolefin/Styrene block |
| | copolymer, 200 μm |
| Tube | Tube: APP 107, 1000 μm |
| Twist-off/ | Polypropylene twist-off/Injection stopper |
| Injection stopper | |

### E. STERILIZATION

General procedures for filling the liquid formulation into the polymeric infusion containers and their processing include aseptic procedures using materials and procedures for aseptic sterilization as known in the art. Alternatively, a composition of the invention comprising sodium bicarbonate without dextrose can be terminally sterilized within the container. For example, the container filled with a composition of the invention can be autoclaved.

Processing techniques of the formulations filled in an infusion container preferably use a sterilization process to destroy or eliminate any microorganisms that may be present in the sodium bicarbonate formulations following preparation. For example, sterilization can be used to destroy or eliminate any microorganisms that may be present in the sealed polymeric infusion container containing the sodium bicarbonate liquid formulation.

In certain embodiments, the liquid formulation containing sodium bicarbonate is substantially free of viable microbial contamination after sterilization. Methods for assessing microbial contamination are known in the art and include testing for sterility in accordance with USP <71> using Trypticase Soy Broth (Soybean-Casein Digest Medium), which is suitable for the culture of both fungi and aerobic bacteria and/or Fluid Thioglycolate Medium, which is a medium primarily intended for the culture of anaerobic bacteria.

Filtration and filling may occur in a controlled environment.

### G. STABILITY OF FINAL PACKAGED PRODUCT IN INFUSION BAG

Advantageously, the liquid sodium bicarbonate formulation of the invention and containment in a polymeric infusion administration bag, provides extended stability and preservation of the liquid sodium bicarbonate formulation of the invention.

Exemplary release specifications for sodium bicarbonate are at least 98%, at least 99%, and at least 99.9% of the specified amount of sodium bicarbonate (e.g., 12.6 mg/mL).

Exemplary release specifications for related compounds, such as any impurities like aluminum content is not more than 25 μg/L. More preferably, the sodium bicarbonate composition of the invention has an aluminum content of not more than 24 mcg/L, or not more than 23 mcg/L, or not more than 22 mcg/L, or not more than 21 mcg/L, or not more than 20 mcg/L, or not more than 19 mcg/mL, or not more than 18 mcg/mL, or not more than 17 mcg/mL, or not more than 16 mcg/ml. or not more than 15 mcg/L, or not more than 14 mcg/mL, or not more than 13 mcg/mL, or not more than 12 mcg/mL, or not more than 11 mcg/mL, or not more than 10 mcg/L, or not more than 9 mcg/mL, or not more than 8 mcg/L, or not more than 7 mcg/L, or not more than 6 mcg/L, or not more than 5 mcg/L, aluminum.

Stability of a sterilized sodium bicarbonate liquid formulation packaged in a polymeric infusion container such as an IV bag may be assessed following storage for at least two weeks, at least 1 month, at least 2 months, at least 3 months, at least 6 months, at least 12 months, at least 15 months, at least 18 months, or at least 24 months. In particular, pH, osmolality, sodium bicarbonate concentration, other impurities, may be assessed after storage for about 1, 2, 3, 6, 9, 12, 15, 18, 24, 36, and/or 48 months. Storage conditions may be long term, intermediate, or accelerated conditions. In particular, storage conditions may be, for example, 25° C.±2° C. and 40% relative humidity (RH)±5% RH, 30° C.±2° C. and 65% RH±5% RH, and/or 40° C.±2° C. and not more than 25% RH.

It is preferred that the compositions presented herein are stored in an autoclavable flexible polymeric container. Notably, certain polymeric materials can affect chemical and storage stability. For example, some polymeric materials are prone to produce higher quantities of crystallized material and/or precipitates (e.g., at an average particle size of about 10 micron or 25 micron), whereas certain other materials were unexpectedly prone to water loss due to water vapor permeability, particularly at extended periods of storage (e.g., one to several months). Therefore, a secondary cover or overwrap of the polymeric material can be employed to maintain predetermined concentration of all solutes. Among other materials as are known in the art, particularly preferred overwrap materials comprise polymeric composite films with at least one metal (e.g., aluminum) layer.

With respect to suitable polymeric autoclavable materials, preferred polymeric materials include polysulfone, polycarbonate, polypropylene, polyethylene (LDPE or HDPE), ethylene/propylene copolymers, polyolefins, acrylic-imide copolymers, polyester (e.g., PET, PEN and the like), TEFLON®, NYLON®, acetal (DELRIN®), polymethylpentene, PVDC, ethylvinylacetate, polyvinyl chloride, and polyolefins. Polyolefin containers are preferred because they remain transparent and dimensionally stable, even after autoclaving.

As will be readily appreciated, the polymeric containers can be prepared in numerous forms. However, flexible bags are especially preferred. The volume of the polymeric container may change considerably. However, it is typically preferred that the container has a volume that accommodates the volume of solution required for a single and complete administration of the solutions presented herein. Therefore, a particularly preferred internal volume of the container is 100 mL, or 250 mL, or 500 mL, or 1000 mL (1 L) or 1500 mL (1.5 L), or 2000 mL (2 L). Moreover, it should be appreciated that the polymeric container will have a single or a double port that is typically sealed with a stopper as is well known in the art

In certain embodiments, storage of the sterilized sodium bicarbonate liquid formulation packaged in a polymeric infusion container is at 25° C.±2° C. and 40%±5% relative humidity for between about 1 month and about 48 months, between about 2 months and about 36 months, or between about 3 months and about 24 months. In certain embodiments, storage of the terminally sterilized sodium bicarbonate liquid formulation packaged in a polymeric infusion container is at 25° C.±2° C. and 40%±5% relative humidity for at least 3 months, at least 6 months, at least 12 months, at least 15 months, at least 18 months, or at least 24 months. In some such embodiments, storage stability is assessed at about 3, 6, 9, 12, 15, 18, 24, and/or 36 months.

In certain embodiments, storage of the terminally sterilized sodium bicarbonate liquid formulation packaged in a polymeric infusion container is at 30° C.±2° C. and 65%±5% relative humidity for between about 1 month and about 24 months or between about 1 month and about 12 months. In certain embodiments, storage of the terminally sterilized sodium bicarbonate liquid formulation packaged in a polymeric infusion container is at 30° C.±2° C. and 65%±5% relative humidity for at least 2 months, at least 3 months, at least 6 months, or at least 12 months. In some such embodiments, storage stability is assessed at about 1, 2, 3, 6, and/or 12 months.

In certain embodiments, storage of the terminally sterilized sodium bicarbonate liquid formulation packaged in a polymeric infusion container is at 40° C.±2° C. and not more than 25% relative humidity for between about 1 month and about 12 months or between about 1 month and about 6 months. In certain embodiments, storage of the terminally sterilized sodium bicarbonate liquid formulation packaged in a polymeric infusion container is at 40° C.±2° C. and not more than 25% relative humidity for at least 2 months, at least 3 months, or at least 6 months. In some such embodiments, storage stability is assessed at about 1, 2, 3, and/or 6 months.

Storage stability assessments may include pH, osmolality, sodium bicarbonate concentration, other impurities.

In certain embodiments, the sterilized sodium bicarbonate liquid formulation packaged in a polymeric infusion container such as an IV bag has a pH in the range of about 7.0 to about 8.5, preferably, from about 7.0-7.9 after storage for up to 6 months, up to 9 months, up to 12 months, up to 15 months, up to 18 months, or up to 24 months under long-term stability conditions.

In certain embodiments, the pH of the sterilized sodium bicarbonate liquid formulation packaged in a polymeric infusion container such as an IV bag does not materially change over time under long-term stability conditions.

In certain embodiments, the sterilized sodium bicarbonate liquid formulation packaged in a polymeric infusion container such as an IV bag retains at least 90%, alternatively at least 91%, alternatively at least 92%, alternatively at least 93%, alternatively at least 94%, alternatively at least 95%, alternatively at least 96%, alternatively at least 97%, or alternatively at least 98% of the sodium bicarbonate in the liquid formulation after storage for up to 6 months, up to 9 months, up to 12 months, up to 15 months, up to 18 months, or up to 24 months under long-term stability conditions as determined by HPLC.

In some such embodiments, the amount of sodium bicarbonate retained following storage is with reference to the sodium bicarbonate concentration prior to storage. In some such embodiments, the sodium bicarbonate concentration prior to storage is from about 1.0% to about 1.5%, preferably from about 1.1% to about 1.4%, more preferably from about 1.2% to about 1.3%, and most preferably from about 1.25% to about 1.27%. In some such embodiments, the sodium bicarbonate concentration prior to storage is 1.26%.

In certain embodiments, the amount of sodium bicarbonate in the sterilized sodium bicarbonate liquid formulation packaged in a polymeric infusion container such as an IV bag does not materially change over time under long-term stability conditions.

In certain embodiments, the sterilized sodium bicarbonate liquid formulation packaged in a polymeric infusion container such as an IV bag contains not more than not more than 25 μg/L, not more than 20 μg/L, not more than 15 μg/L, not more than 10 μg/L, not more than 5 μg/L, of Aluminum content after storage for up to 6 months, up to 9 months, up to 12 months, up to 15 months, up to 18 months, or up to 24 months under long-term stability conditions as determined by ICP-MS.

In certain embodiments, the liquid formulation is stable for at least 12 months or at least 18 months when stored at 25° C.±2° C. and 40% relative humidity (RH)±5% RH. In some such embodiments, the liquid formulation is stable for up to 24 months when stored at 25° C. 2° C. and 40% relative humidity (RH)±5% RH.

In some such embodiments, the liquid formulation retains at least 90% w/w, preferably at least 95% w/w, and more preferably at least 98% of the sodium bicarbonate after storage at 25° C.±2° C. and 40% RH±5% RH for 12, 18, or 24 months.

In some such embodiments, the liquid formulation retains at least 90% w/w, preferably at least 95% w/w, and more preferably at least 98% of the sodium bicarbonate after storage at 25° C.±2° C. and 40% RH±5% RH for 12, 18, or 24 months.

### H. METHODS OF USE

In at least one aspect, this disclosure provides methods for the management of acidosis, e.g., metabolic acidosis or ketosis using the liquid formulations described herein. Liquid formulations comprising sodium bicarbonate may be used for the management of drug overdose that can be treated with an alkalinization of the blood or urine. Liquid formulations described herein may preferably be parenterally administered to a subject in need thereof. In particular, liquid formulations described herein may be intravenously administered to a subject in need thereof, preferably at a determined infusion rate.

Thus, in certain embodiments, the methods comprise parenterally administering to a subject in need of reversal of acidosis, the sterilized sodium bicarbonate liquid formulations described herein. In some such embodiments, the methods comprise intravenously administering or administering by infusion to a subject in need of management of acidosis the sterilized sodium bicarbonate liquid formulations described herein.

In certain embodiments, the subject is a human.

The liquid formulations, methods, and uses described herein are included as an illustration of and not a limitation upon the scope of the invention.

The above disclosure and example generally describe the present invention and is provided for purposes of illustration and is not intended to limit the scope of the invention. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A stable, ready-to-administer parenteral liquid formulation consisting of 1.0% to about 1.5% sodium bicarbonate dissolved in CO₂-bubbled water for injection
wherein the liquid formulation has 25 meg/L or less of aluminum;
wherein the liquid formulation is without dextrose;
wherein the liquid formulation is isotonic;
wherein the liquid formulation has a pH of 7.0 to 8.5
wherein the liquid formulation is contained in a polymeric infusion container;
wherein the liquid formulation is stable for at least 6 months when stored at 25° C.±2° C. and 40% relative humidity (RH)±5% RH; and
wherein the liquid formulation is terminally sterilized within the polymeric infusion container by heat.

2. The liquid formulation of claim 1, wherein the liquid formulation has a pH of 7.0-7.9.

3. The liquid formulation of claim 1, wherein the liquid formulation comprises about 1.2% to about 1.3% sodium bicarbonate.

4. The liquid formulation of claim 1, wherein the liquid formulation comprises about 1.26% w/v of sodium bicarbonate.

5-9. (canceled)

10. The liquid formulation of claim 1, wherein the liquid formulation retains at least 98% w/w of the sodium bicarbonate and has not more than 25 μg/L of Aluminum content after storage at 25° C.±2° C. and not more than 40% RH for at least 6 months.

11. The liquid formulation of claim 1, wherein the liquid formulation retains at least 99% w/w of the sodium bicarbonate and has not more than 15 μg/L of Aluminum content after storage at 25° C.±2° C. and not more than 40% RH for at least 6 months.

12. The liquid formulation of claim 1, wherein the liquid formulation retains at least 99.9% w/w of the sodium bicarbonate after storage at 25° C.±2° C. and not more than 40% RH for at least 6 months.

13. A ready-to-administer parenteral liquid formulation consisting of 12.6 mg/mL of sodium bicarbonate dissolved in CO₂-bubbled water for injection;
wherein the liquid formulation has 25 mcg/mL or less of aluminum;
wherein the liquid formulation is isotonic and is without dextrose;
wherein the liquid formulation has a pH of 7.0 to 8.5,
wherein the liquid formulation is contained in a polymeric infusion container and terminally sterilized by heat within the polymeric infusion container, and
wherein the liquid formulation is stable for at least 6 months when stored at 25° C.±2° C. and not more than 40% RH.

14. The liquid formulation of claim 13, wherein the liquid formulation has a pH of 7.0 to 7.9.

15-17. (canceled)

18. A method of treating a patient in need of management of acidosis comprising parenterally administering to the patient the liquid formulation of claim 1.

19. A method of treating a patient in need of management of acidosis comprising parenterally administering to the patient the liquid formulation of claim 13.

20. A polymeric infusion container filled with the liquid formulation selected from the group consisting of liquid formulation of claim 1 and the liquid formulation of claim 13.
